(19) United States
(12) Patent Application Publication
(10) Pub. No.: US 2014/0228715 A1
(43) Pub. Date: Aug. 14, 2014

(54) Method and Apparatus for Delivering a Substance

(71) Applicants: The General Hospital Corporation, Boston, MA (US); Massachusetts Institute of Technology, Cambridge, MA (US)

(72) Inventors: Avraham D. Schroeder, Newton, MA (US); Carlo Giovanni Traverso, Cambridge, MA (US); Baris E. Polat, Brighton, MA (US); Kathryn A. Whitehead, Somerville, MA (US); Daniel G. Anderson, Sudbury, MA (US); Robert S. Langer, Jr., Newton, MA (US); Carl Magnus Schoellhammer, Santa Monica, CA (US); Daniel Blankschtein, Brookline, MA (US)

(73) Assignees: The General Hospital Corporation, Boston, MA (US); Massachusetts Institute of Technology, Cambridge, MA (US)

(21) Appl. No.: 14/116,628

(22) PCT Filed: May 14, 2012
(86) PCT No.: PCT/US2012/037848

§ 371 Date: Nov. 8, 2013

Related U.S. Application Data
(60) Provisional application No. 61/485,957, filed on May 13, 2011.

(51) Int. Cl. A61N 7/00

(52) U.S. Cl. CPC A61N 7/00
USPC 601/2

(57) ABSTRACT

A method of delivering a substance includes providing a substance at a location in a gastrointestinal (GI) tract, excluding a buccal membrane, of a biological body; and applying ultrasonic waves, having a frequency between about 20 kHz and about 10 MHz, at the location. The method can include storing the substance in at least one reservoir and exposing a medium within or of the GI tract to the substance. The method can further include delivering a device into the GI tract, the device including at least one ultrasound transducer and circuitry; powering the at least one ultrasound transducer and circuitry from within the device; and driving the at least one ultrasound transducer, using the circuitry, in a manner causing the at least one ultrasound transducer to emit ultrasonic waves to a medium within or of the GI tract.

### RELATED APPLICATION

This application claims the benefit of U.S. Provisional Application No. 61/485,957 filed on May 13, 2011.

The entire teachings of the above application are incorporated herein by reference.

### BACKGROUND

Oral administration remains the most commonly used and accepted route for the delivery of drugs. The revolution in drug-development has introduced macromolecules, including proteins (e.g., monoclonal antibodies), as therapeutics, although these are generally limited by their administration to the intravenous or subcutaneous routes.

Ultrasound has had decades of clinical success in diagnostic imaging, blood flow analysis, kidney stone disruption, and tumor and fibroid ablation. In recent years, ultrasound has been used in the clinic to transiently increase the permeability of the skin, enabling transdermal administration of drugs and sampling of extracellular analytes. Since then, ultrasound in the range of 0.02-3 MHz has been shown to improve the delivery of many macromolecules at therapeutic levels, including proteins, nanoparticles, and vaccines. Furthermore, ultrasound has been used to increase the permeability of a variety of biological barriers, such as ocular and buccal tissues and the blood-brain barrier.

### SUMMARY

A method of delivering a substance includes providing a substance at a location in a gastrointestinal (GI) tract, excluding a buccal membrane, of a biological body and applying ultrasonic waves, having a frequency between about 20 kHz and about 10 MHz, at the location.

The method may further include storing the substance in at least one reservoir; and exposing a medium within or of the GI tract to the substance. As used herein, reservoir can include a volume or a coating. For example, the reservoir can be an internal reservoir or volume of a device or a coating of a housing of a device. In an embodiment, the method includes imparting energy into the reservoir to trigger exposure of the medium to the substance. Energy can include ultrasonic waves, light, mechanical vibration, etc. Alternatively or in addition; the method may include emitting the ultrasonic waves to increase passage of the substance through a surface of the GI tract, such as gastrointestinal mucosa. Further, the method can include emitting the ultrasonic waves to facilitate passage of the substance between the at least one reservoir and the medium. For example, the substance may be an analyte released from the biological body with the ultrasonic waves emitted. In some embodiments, the substance passes from the medium into the at least one reservoir.

In an embodiment, exposing the medium to the substance includes pumping the substance between the at least one reservoir and the medium. In another embodiment, exposing the medium to the substance includes allowing the substance to diffuse between the at least one reservoir and the medium. The method may further include propelling the substance towards a surface of the GI tract with the ultrasonic waves. The method may include acoustic streaming or cavitation to enhance update of the substance by tissue of the GI tract.

In an embodiment, the method includes sensing a property of the biological body or energy presented thereto to determine proximity to the location, and applying the ultrasonic waves in response to the property sensed. The property sensed may be pH, light intensity, temperature, the absence or presence of a chemical, the absence or presence of blood, the absence or presence of a hormone, or the absence or presence of an inflamed fluid.

The ultrasonic waves can be applied in a frequency range of from about 20 kHz to about 100 kHz, or from 20 kHz to about 500 kHz, or from about 500 kHz to about 1 MHz, or from about 1 MHz to about 3 MHz, or from about 3 MHz to about 7 MHz, or from about 7 MHz to about 10 MHz. The intensity of the ultrasonic waves applied can be in a range from about 0.1 W/cm2 to about 10 W/cm2, from about 0.24 W/cm2 to about 1.4 W/cm2, from about 1.4 W/cm2 to about 10 W/cm2, from about 10 W/cm2 to about 100 W/cm2, from about 100 W/cm2 to about 500 W/cm2, or from about 500 W/cm2 to about 1000 W/cm2.

The location at which the ultrasonic waves are applied can be within an anatomic location of the GI tract, e.g., stomach, small intestine, large intestine (colon), rectum, or at a duct that enters the GI tract, such as a pancreatic duct or a common bile duct. In a preferred embodiment, the location is in a colon. Applying the ultrasonic waves may increase permeability of tissue at the location. Further, the method may include tuning the ultrasonic waves to increase or decrease enzymatic activity in the GI tract while operating therein. For example, applying the ultrasonic waves may be performed in a manner expected to decrease enzymatic activity to decrease or prevent degradation of the substance.

The substance to be delivered can include a compound having a molecular weight in a range from about 0.1 kDa to about 1000 kDa. The substance can include at least one of the following: a therapeutic compound, a bioactive compound, an imaging agent, a diagnostic agent, or combination thereof. A therapeutic compound may include a polynucleotide, small molecule, peptide, or protein. An imaging agent may include a contrast agent, e.g., a radioactive or contrast dye. In some embodiments, the substance includes a therapeutic compound that includes DNA or RNA. For example, the RNA can be small interfering RNA (siRNA) or micro-RNA (miRNA). In an embodiment, the substance includes a protein, e.g., an antibody.

In an embodiment, the substance to be delivered includes at least one of the following: liposomes, microparticles, nanoparticles, iron oxide particles, gold particles, drug crystals, polymeric particles, lipid or lipid-like particles, or combination thereof. Alternatively or in addition, the substance can include an ultrasound absorbing material.

In some embodiments, applying the ultrasonic waves includes applying the ultrasonic waves at a frequency and intensity known to treat a medical condition through direct stimulus of the ultrasonic waves. For example, the ultrasonic waves applied can include high intensity focused ultrasound (HIFU), which can treat tissue having the medical condition. Applying the ultrasonic waves can include heating, ablating, denaturing, scarring, vibrating, or combinations thereof, of tissue at the location. For example, ultrasound stimulation of tissue can include stimulation of nerves, and may include ultrasound induced vibratory stimulation.

In some embodiments, the method further includes delivering a device into the GI tract, the device including at least one ultrasound transducer and circuitry; powering the at least one ultrasound transducer and circuitry from within the device; and driving the at least one ultrasound transducer, using the circuitry, in a manner causing the at least one ultrasound transducer to emit ultrasonic waves to a medium within or of the GI tract. The device can include a housing with holes defined therethrough, and providing the substance can include exposing the substance via the holes. In an embodiment, the at least one reservoir is associated with the housing of the device.

In some embodiments, the method of delivering a substance includes propelling the device within the GI tract with the ultrasonic waves emitted or other ultrasonic waves emitted by the device.

Delivering the device can include rectally or orally introducing the device into the GI tract. Delivering the device can also include placing the device at a duct that enters the GI tract and activating the ultrasound transducer to increase passage of the substance into tissue of the duct. In an embodiment, delivering the device includes placing the device in the GI tract and activating the ultrasound transducer to increase passage of a substance into tissue of the GI tract. Also, a combination of several devices may be delivered. Further, the ultrasound device, e.g., an ingestible ultrasound pill, may be delivered in addition to another therapeutic compound that a patient ingests or consumes separately, or that a healthcare provider administers separately to the patient.

In some embodiments, the method further includes positioning the device in the GI tract, for example, by employing a positioning mechanism to hold the device stationary. The positioning mechanism can include a suture, e.g., a biodegradable suture, or an externally-placed magnet. In some embodiments, positioning the device includes causing the device to adhere to tissue of the GI tract. For example, the device can include a bioadhesive coating, and causing the device to adhere to the tissue of the GI tract can include using the bioadhesive coating. Alternatively or in addition, causing the device to adhere to the tissue of the GI tract or include releasing an adhesive from a reservoir of the device.

A device includes a housing configured to be delivered into a gastrointestinal (GI) tract of a biological body and at least one ultrasound transducer, disposed within the housing. The ultrasound transducer is configured to emit ultrasonic waves, external from the housing, within a frequency range of from about 20 kHz to about 10 MHz to a medium within or of the GI tract. Also included is circuitry, disposed within the housing and configured to drive the transducer; and a power source, disposed within the housing and configured to power the transducer and the circuitry.

In an embodiment, the device further includes at least one reservoir, e.g., volume or coating, associated with the housing and configured to store the substance to be delivered; and a mechanism, associated with the housing and configured to expose the medium to the substance. The mechanism may be configured to be activated by energy imparted into the reservoir to trigger exposure of the medium to the substance. In an embodiment, the substance is released from the reservoir by ultrasound imparted into the reservoir. Alternatively or in addition, the mechanism can include material sensitive to pH or temperature to trigger exposure of the medium to the substance. In some embodiments, the mechanism includes a pump, which may be a mechanical, chemical, osmotic pump. For example, the power source can include a deformable battery configured to serve as the pump and the power source. In an embodiment, the housing defines holes therethrough and the mechanism is configured to expose the medium to the substance via the holes. The mechanism can also be configured to utilize a concentration gradient between the reservoir and the medium external from the housing to transfer the substance by diffusion. In some embodiments, the housing is ingestible. The housing may also be implantable.

In some embodiments, the device further includes a sensor disposed within the housing and coupled to the circuitry, the sensor being configured to activate the at least one ultrasound transducer. The sensor can be a pH sensor, electromagnetic wave sensor, light sensor, temperature sensor, or chemical sensor, and may be configured to trigger release of the substance from a reservoir associated with the housing.

The ultrasound transducer of the device can be configured to operate in a frequency range of from about 20 kHz to about 100 kHz, or from about 20 kHz to about 500 kHz, or from about 500 kHz to about 1 MHz, or from about 1 MHz to about 3 MHz, or from about 3 MHz to about 7 MHz, or from about 7 MHz to about 10 MHz. The ultrasound transducer can be configured to emit ultrasonic waves to the medium at an intensity in a range from about 0.1 W/cm² to about 10 W/cm², from about 0.24 W/cm² to about 1.4 W/cm², from about 1.4 W/cm² to about 10 W/cm², from about 10 W/cm² to about 100 W/cm², from about 100 W/cm² to about 500 W/cm², from about 500 W/cm² to about 1000 W/cm².

Further, the device can be calibrated to emit ultrasonic waves to increase or decrease enzymatic activity in the body lumen. In an embodiment, the power source can be a battery configured to be recharged through a wireless connection with a device external from the body.

In some embodiments, the device is configured to be positioned in the GI tract and held stationary using a positioning mechanism, such as a suture or an externally-placed magnet. In some embodiments, the device is configured to adhere to tissue of the GI tract. For example, the device can include a bioadhesive coating, and the device can be configured to adhere to the tissue of the GI tract through the use of the bioadhesive coating. Alternatively or in addition, the device can include an adhesive releasable from a reservoir of the device.

In an embodiment, the at least one ultrasound transducer is configured to emit the ultrasonic waves in the presence of the substance to be delivered at a location of the GI tract.

In an embodiment, the device includes at least one reservoir associated with the housing, and a mechanism, associated with the housing and configured to transfer the substance between the medium and the at least one reservoir.

A method of treating inflammatory bowel disease includes providing a self-powered ultrasound device comprising at least one reservoir, each reservoir comprising a substance that includes a therapeutic molecule for treatment of inflammatory bowel disease; releasing the substance from the at least one reservoir into the gastrointestinal tract in the vicinity of target tissue for localized delivery of the substance; and emitting ultrasonic waves in a frequency range of from about 20 kHz to about 10 MHz with the ultrasound device to increase passage of the substance through a surface of the target tissue.

In an embodiment, the substance further includes nutrients. In some embodiments, emitting the ultrasonic waves further includes emitting the ultrasonic waves with the ultrasound device to increase permeability of the target tissue to the substance. The target tissue can include gastrointestinal mucosa and may include colon tissue.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a diagram of a digestive system of a biological body illustrating delivery of an ultrasound device into a gastrointestinal (GI) tract.

FIG. 2 illustrates positioning of a device in the GI tract using a suture or an adhesive.

FIG. 3 illustrates positioning of a device in the GI trace using an externally-placed magnet.

FIG. 4 is a schematic diagram illustrating an embodiment of an ultrasound device for delivery in to the GI tract.

FIG. 5 is a schematic diagram illustrating another embodiment of an ultrasound device for delivery into the GI tract.

FIG. 6 is a schematic diagram of an experimental system to evaluate the enhancement of the permeability of a GI tract membrane by exposure to ultrasound.

FIG. 7 is a graph illustrating enhancement of sucrose penetration into porcine small intestine due to exposure to 1-Mhz or 3-MHz ultrasound at different amplitudes.

FIG. 8 is a graph illustrating enhanced penetration of ¹⁴C-inulin into porcine small intestine measured after exposure to 1-MHz ultrasound (1.4 W/cm²) at different irradiation profiles. Black tiles represent the enhancement of inulin uptake in the tissue, while the white tiles represent the enhancement % of transport of inulin across the tissue.

FIG. 9 is a graph illustrating enhanced penetration of 70 kDa ¹⁴C-dextrose into porcine small intestine after exposure to 1-MHz ultrasound (1.4 W/cm²) at different irradiation profiles. Black tiles represent the enhancement of inulin uptake in the tissue, while the white tiles represent the enhancement percentage of transport of inulin across the tissue.

FIG. 10 is an image of an embodiment of an ultrasound device in a fluid showing the device in the “off” state, not emitting ultrasound, and a model tracer (dye) injected in the fluid.

FIG. 11 is an image of the device of FIG. 10 showing the device in the “on” state, emitting ultrasound and propelling the tracer through the fluid.

FIG. 12A is an image of the device of FIG. 10 in a colon recorded with an external ultrasound imaging apparatus.

FIG. 12B is an image of the device of FIG. 10 in a colon illustrating the interference pattern induced by the ultrasound being emitted from the device.

FIG. 13 is a graph illustrating enhancement of delivery of a radioactive tracer (glucose) to tissue of the small intestine using ultrasound.

FIG. 14 is a graph illustrating enhancement of delivery of a radioactive tracer (glucose) to tissue of the colon using ultrasound.

### DETAILED DESCRIPTION OF EMBODIMENTS OF THE INVENTION

Ultrasound is widely used across medical disciplines. Recently, ultrasound has been approved by the Federal Drug Administration (FDA) as a tool to enhance transdermal drug delivery. Conventional ultrasound devices, which include a control unit, a probe, and an external power supply, are relatively large. Described herein are embodiments of a delivery device that includes an ingestible/implantable capsule or pill which houses an entire ultrasound apparatus. Such a stand-alone, self-powered ultrasonic device can be used for localized delivery of therapeutic macromolecules via the gastrointestinal (GI) tract. Recent technological advancements in miniaturization include high efficiency battery-power usage and novel embedding of the electronics on a microchip. An embodiment of the self-standing ultrasound pill has dimensions that can be introduced into the mouth, swallowed, and transported through the digestive tract for drug delivery applications.

Miniaturizing ultrasonic devices was noted with the introduction of the hand-held ultrasound diagnostic imaging systems by Siemens and GE in 2007. Further miniaturization of ultrasonic devices, is enabled by optimizing power transfer and efficiency. Using low output impedance amplifier circuits to replace RF amplifiers and impedance-matching circuitry increases the power transferred from the battery to the transducer to more than 95%. The first device to utilize this advancement was an implantable transducer, in dimensions of several centimeters, aimed at delivering drugs to brain tumors.

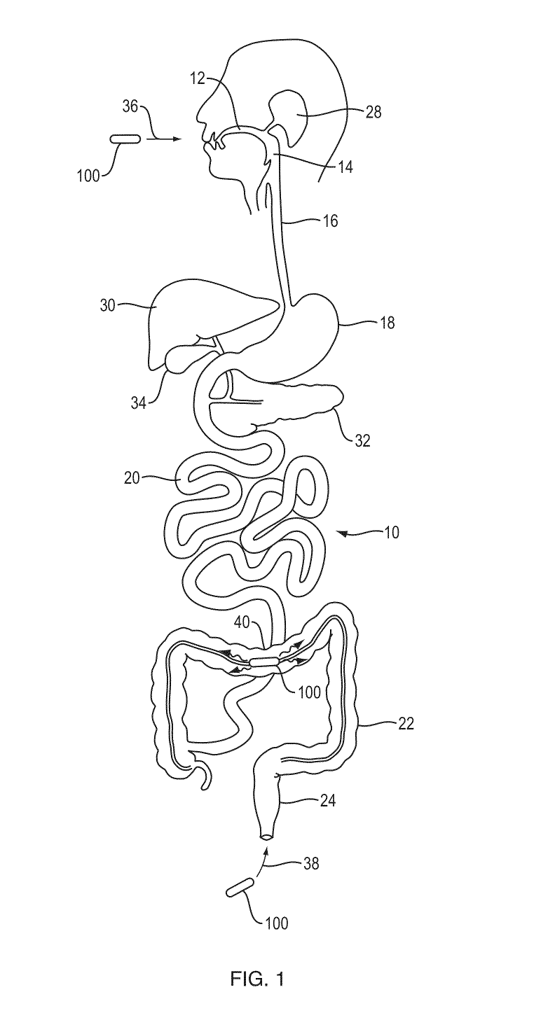

FIG. 1 is a diagram of a digestive system illustrating delivery of an ultrasound device 100 into a GI tract 10. The figure shows various parts of the GI tract 10, including mouth 12, pharynx 14, esophagus 16, stomach 18, small intestine 20, large intestine or colon 22, and rectum 24. The mouth 12 includes the tongue and the buccal membranes (not shown). The term digestive tract is sometimes used synonymously with GI tract. Typically, the term digestive tract is used to denote the GI tract and ancillary organs and structures, such as salivary glands 28, liver 30, pancreas 32, and gallbladder 34, including ducts connecting various organs, such as the pancreatic and common bile ducts. Device 100 may be introduced orally or rectally, as shown by arrows 36 and 38, respectively, and may also be otherwise placed in the GI tract 10. In general, one or more devices 100 can be used to deliver a substance and apply ultrasound at a location in the GI tract. As shown at 40, the location can be in a colon 22.

In an embodiment, a method of delivering a substance includes providing a substance to a location in the GI tract 10, excluding a buccal membrane, of a biological body and applying ultrasonic waves, having a frequency between about 20 kHz and about 10 MHz, at the location. Providing the substance and applying the ultrasound may occur simultaneously or may be offset in time, and may be adjusted according to different dosing regimens. For example, the method can include pre-administration of the substance followed by the application of the ultrasonic waves, coincident administration/application of the substance and the ultrasonic waves, and application of the ultrasonic waves followed by administration of the substance.

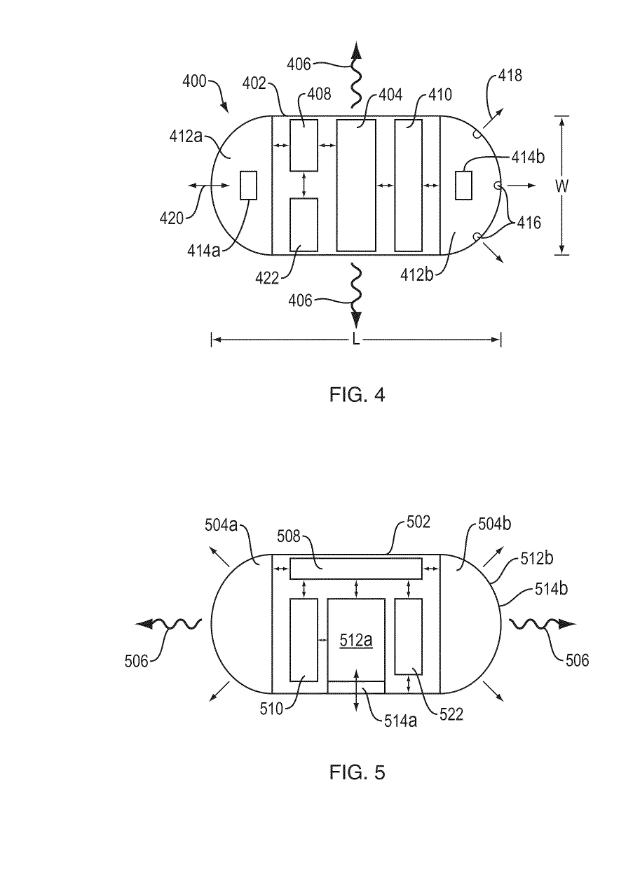

The method may further include storing the substance in at least one reservoir, e.g.; of device 100, and exposing a medium within or of the GI tract, e.g. fluid or tissue, to the substance. For example, the reservoir can be an internal reservoir or a coating, as described in more detail with reference to FIGS. 4 and 5. Energy, such as ultrasonic waves, light, mechanical vibration, etc., may be imparted into the reservoir of device 100 to trigger exposure of the medium to the substance. The ultrasonic waves may be emitted to increase passage of the substance through a surface, e.g., internal surface, of the biological body, such as GI mucosa. Further, the method can include emitting the ultrasonic waves to facilitate passage of the substance, or another substance or fluid, between the at least one reservoir and the medium. For example, the substance may be an analyte released from the biological body with the ultrasonic waves emitted. In some embodiments, the substance, or another substance or fluid, passes from the medium into the at least one reservoir.

A property of the biological body, or energy presented thereto, may be sensed to determine proximity of device 100 to the location. Ultrasonic waves may then be applied in response to the property sensed. For example, the device 100 may include a sensor that senses pH as the device 100 passes through the GI tract 10. It is generally understood that the pH varies for different locations in the GI tract. Typically, the stomach has a lower pH, e.g., pH=1.3, as compared to the small intestine, whose pH can vary along its length from pH=6, in the duodenum, to pH=7.8, in the jejunum and ileum. Other properties sensed may include light intensity, temperature, the absence or presence of a chemical, the absence or presence of blood, the absence or presence of a hormone, or the absence or presence of an inflamed fluid.

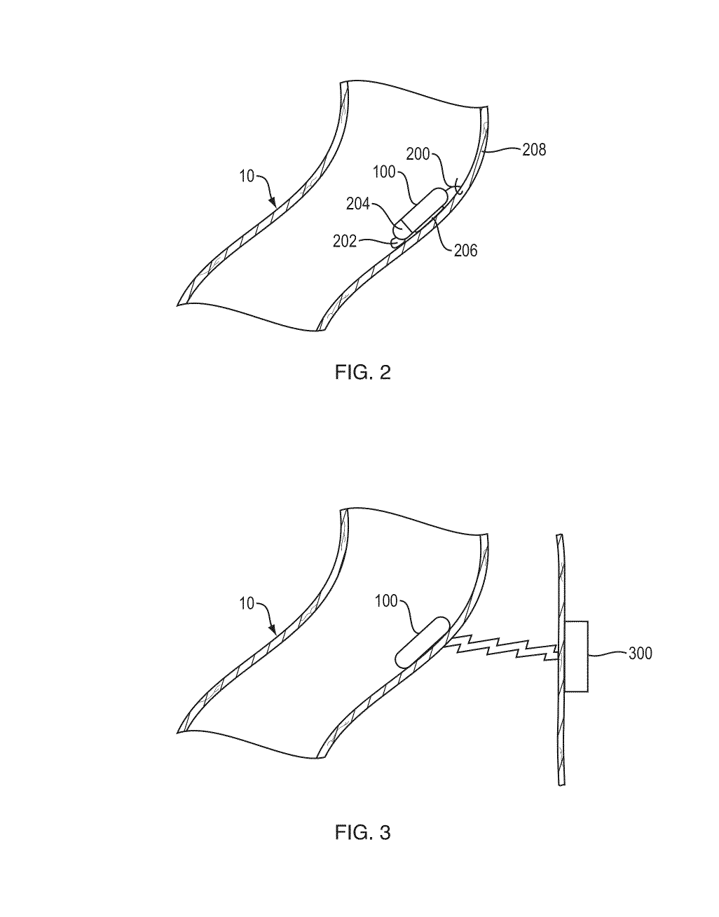

In some embodiments, providing a substance includes positioning the device 100 in the GI tract, for example, by employing a positioning mechanism to hold the device stationary. As described with references to FIGS. 2 and 3 below, the positioning mechanism can include a suture, e.g., a biodegradable suture, or an externally-placed magnet. In some embodiments, positioning the device includes causing the device to adhere to tissue of the GI tract. The positioning mechanism can be used to facilitate applying ultrasound at a specific location and for a specific duration.

FIG. 2 illustrates positioning of device 100 in the GI tract 10 using a suture 200. Suture 200 may be used to engage tissue 208, e.g. mucosa, to hold device 100 in place. Alternatively or in addition, the device 100 can adhere to the tissue 208 of the GI tract by releasing an adhesive 202 from a reservoir 204 of the device 100. Further, the device 100 can include a bioadhesive coating 206 that causes the device to adhere to the tissue 208 of the GI tract.

FIG. 3 illustrates positioning of device 100 in the GI tract 10 using an externally-placed magnet 300. For example, magnet 300 can be used to hold the device 100 stationary at a location in the GI tract for localized delivery of a substance, localized application of ultrasound, or both.

FIG. 4 is a schematic diagram illustrating an example ultrasound device 400. The device 400 includes a housing 402, configured to be delivered into a GI tract 10 of a biological body, and at least one ultrasound transducer 404, disposed within the housing 402. The ultrasound transducer 404 is configured to emit ultrasonic waves 406, external from the housing, within a frequency range of from about 20 kHz to about 10 MHz. As shown, the ultrasonic waves can be emitted at the sides of the housing 402 and may be emitted radially. The transducer is configured to emit sound waves to a medium within or of the GI tract 10, for example, in the presence of a substance to be delivered at a location of the GI tract. The device 400 can propel the substance towards a surface of the biological body, e.g., a surface of the GI tract, with the ultrasonic waves emitted. Device 400 includes circuitry 408, disposed within the housing 402 and configured to drive the transducer 404. A power source 410 is disposed within the housing 402 and configured to power the transducer 404 and the circuitry 408. The power source may be a battery and may be rechargeable.

The device 400 further includes at least one reservoir, e.g., volume or coating, associated with the housing 402 and configured to store the substance. As shown in FIG. 4, the device 400 includes reservoirs 412a and 412b (collectively 412a-b), and mechanisms 414a and 414b (collectively 414a-b), associated with the housing 402 and configured to expose the medium to the substance. The mechanism 414a-b may be configured to be activated by energy imparted into the reservoir 412a-b to trigger exposure of the medium to the substance. Alternatively or in addition, the mechanism 414a-b can include material sensitive to pH or temperature to trigger exposure of the medium to the substance. In some embodiments, the mechanism 414 includes a pump to pump the substance between the at least one reservoir and the medium. The pump may be a mechanical, chemical, osmotic pump. In an embodiment, the housing 402 defines holes 416 therethrough, where the holes may be configured to allow passage of the substance (or an external substance or fluid) responsive to activity or effects thereof by the pump or sound waves internally or externally. The mechanism, e.g., mechanism 414b, can be configured to expose the medium to the substance via the holes 416 as shown at 418. The mechanism can also be configured to utilize a concentration gradient between the reservoir, e.g., reservoir 412a, and the medium external from the housing 402 to transfer the substance by diffusion as shown at 420. The substance may be an analyte released from the biological body with the ultrasonic waves emitted. In some embodiments, the substance passes from the medium into the reservoir 412. Multiple reservoirs 412a and 412b may store multiple substances. For example, a first reservoir 412a may store an analyte and a second reservoir 412b may store a drug or other substance for delivery into the GI tract.

In some embodiments, the housing 402 is ingestible. The housing 402 may also be implantable. As shown, the housing 402 is in the form of a pill that has a length L, e.g., from about 1 cm to about 3 cm, and a width or diameter W, e.g., from about 0.5 cm to about 1 cm.

The device 400 further can further include a sensor 422 disposed within the housing 402 and coupled to the circuitry 408, the sensor being configured to activate the at least one ultrasound transducer 404. The sensor can be a pH sensor, electromagnetic wave sensor, light sensor, temperature sensor, or chemical sensor, and may be configured to trigger release of the substance from reservoir 414.

It should be understood that the schematic diagram of FIG. 4 (and other figures presented herein) is an example embodiment and that mechanical or electrical interconnectors and orientations between or among components can be modified to produce various effects, such as radial or axial directing of ultrasonic waves.

FIG. 5 is a schematic diagram illustrating another example of an ultrasound device. The device 500 includes a housing 502 configured to be delivered into a GI tract 10 of a biological body and ultrasound transducers 504a and 504b (collectively 504a-b), disposed within the housing 502. The ultrasound transducer 504a-b is configured to emit ultrasonic waves 506, external from the housing 502, within a frequency range of from about 20 kHz to about 10 MHz. As shown, the ultrasonic waves can be emitted at the ends of the housing 502. Similar to the transducer 404 of the device 400 of FIG. 4, the transducer 504a-b is configured to emit sound waves to a medium within or of the GI tract 10, for example, in the presence of a substance to be delivered at a location of the GI tract. The devices 400 and 500 can be configured propel the substance towards a surface of the biological body with the ultrasonic waves emitted. Similar to device 400 of FIG. 4, the device 500 of FIG. 5 includes circuitry 508, disposed within the housing 502 and configured to drive the transducer 504a-b, and a power source 510, disposed within the housing 502 and configured to power the transducer 504a-b and the circuitry 508.

The device 500 further includes an internal reservoir or volume 512a and reservoir or coating 512b. Optionally, only one reservoir may be present. Reservoirs 512a, 512b (collectively 512a-b) are associated with the housing 502 and configured to store one or more substances. For example, a first reservoir 512a can store the substance to be delivered to the medium at the location of the GI tract and a second reservoir 512b can store another substance transferred from the medium to the reservoir, or vice versa.

As shown in FIG. 5, the device 500 includes mechanisms 514a and 514b (collectively 514a-b), associated with the housing 502 and configured to expose the medium to the substance. The mechanism 514a-b may be configured to be activated by energy imparted into the reservoir 512a-b to trigger exposure of the medium to the substance. Alternatively or in addition, the mechanism 514a-b can include material sensitive to pH or temperature to trigger exposure of the medium to the substance. For example, the device 500 may include a coating configured to serve as reservoir 512b for storing the substance and mechanism 514b for exposing the medium to the substance. A shown, the coating may be included in or applied to the housing 502 to cover all or part of the ultrasound transducer 504b. In this way, exposure of the medium to the substance, e.g., release of the substance from the coating, can be triggered via the ultrasonic waves emitted by the transducer 504b into and through the coating. The mechanism 514a, for example, can include a pump, which may be a mechanical, chemical, osmotic pump, to transfer a substance between the medium and the reservoir 512a. Furthermore, the power source 510 can include a deformable battery configured to serve as the pump and the power source. The mechanism 514a-b can also be configured to utilize a concentration gradient between the reservoir 512a-b and the medium external from the housing 502 to transfer the substance by diffusion. As with the housing 402 (FIG. 4), the housing 502 may be ingestible or implantable and can be in the form of a pill that has a length L and width, or diameter, W.

The device 500 can further include a sensor 522 disposed within the housing 502 and coupled to the circuitry 508, the sensor being configured to activate any of the ultrasound transducers 504a and 504b. The sensor 522 can be a pH sensor, electromagnetic wave sensor, light sensor, temperature sensor, or chemical sensor, and may be configured to trigger release of the substance from the reservoir 512a-b.

In the embodiments described herein, such as devices 100, 400, and 500 described with reference to FIGS. 1-5, one or more ultrasound transducer can be configured to operate in a frequency range of from about 20 kHz to about 100 kHz, or from about 20 kHz to about 500 kHz, or from about 500 kHz to about 1 MHz, or from about 1 MHz to about 3 MHz, or from about 3 MHz to about 7 MHz, or from about 7 MHz to about 10 MHz. Furthermore, one or more ultrasound transducers can be configured to emit ultrasonic waves to the medium at an intensity in a range from about 0.1 W/cm² to about 10 W/cm², from about 0.24 W/cm² to about 1.4 W/cm², from about 1.4 W/cm² to about 10 W/cm², from about 10 W/cm² to about 100 W/cm², from about 100 W/cm² to about 500 W/cm², from about 500 W/cm² to about 1000 W/cm².

### Example 1

### Ultrasound Pill Design

In one embodiment, the dimensions of the device are 2.7 cm in length (L) and 1 cm in width or diameter (W). Similar dimensions were previously approved by the FDA for an ingestible pill containing a camera used for diagnostic imaging of the digestive tract. As described elsewhere herein, the device can include a housing in the form of a pill and configured to house all the components needed for producing an ultrasonic signal, such as an ultrasound transducer, a power source, and circuitry connected to the power source and the transducer. The device can further include at least one reservoir and a release mechanism associated with the housing.

The hereby described technology is of a self-standing ultrasound machine in dimensions that can be introduced into the mouth, swallowed, and/or transported through the digestive tract. The technology may be used for, but not confined to:
a) Improving the delivery of substances, such as drugs, to the intestinal mucosa, or to improve the ability of substances to transfer through the mucosa, and/or to improve the delivery of substances to the mouth, by utilizing ultrasound-facilitated increased permeability.
b) Improving ultrasound facilitated imaging by introducing an inner source of ultrasonic waves of known parameter at the source.
c) Using ultrasonic waves to treat medical conditions of the mouth and/or of the digestive tract. A device according to the principles of the invention includes an ultrasonic transducer/s, a power source (such as a battery), a microprocessor to operate the ultrasound transducer and other apparatuses (such as the reservoir and/or sensing devices), a reservoir and apparatus for releasing/uploading materials out-of or into the reservoir, a housing or casing that would enable either adherence/non-adhering to the GI mucosal membrane.

The ultrasound device can be designed to operate at a frequency within the range of 20 kHz to 10 MHz.

### Example (a)

### Delivery of a Substance to the GI Tract

The pill-shaped ultrasound device, loaded with a therapeutic substance, such as insulin, can be administered orally (and swallowed) by the patient. By sensing pH in the GI tract, a sensor in the device can activate the transducer to increase the penetration of macromolecules into the GI mucosa and/or to propel the therapeutic compound towards the mucosa and improve drug delivery. The release of the substance or drug from the internal reservoir can be triggered by pressure (using an internal pump), or by diffusion (utilizing the concentration gradient between the reservoir and the external medium). The substance or drug may be transferred through holes in the house, or holes in the transducer ceramics, and out of the pill in order to improve drug propulsion.

### Example (b)

### Increasing Oral Delivery of a Substance

An ultrasound pill comprising a drug can be placed under the tongue. Upon placement, the pill can be activated to increase the permeability of mouth tissue by ultrasonic waves. Ultrasonic waves can also propel the drug from the surface of the device or transducer towards and into the tissue. This device can enable rapid delivery of a drug to into the blood stream, avoiding GI-tract metabolism, and may be used as a replacement of intravenous administration of drugs. The device can also be used for drugs focused at the oral cavity, and or for the administration of pain relievers to the mouth membrane, aside from under the tongue.

### Example (c)

### Affecting Enzymatic Activity in the GI Tract by Ultrasound

Ultrasound has been previously used to affect the activity of enzymes. The ultrasound pill can be calibrated to decrease or increase the activity of enzymes.

For example, when placing the pill under the tongue, or while traveling through the GI tract, ultrasonic waves can be used to decrease enzymatic activity thereby preventing drug degradation.

### Example (d)

### Using an Inner Ultrasonic Signal for Improving Ultrasonic Imaging

Having an inner source of ultrasonic waves, of known parameters at the source (frequency, amplitude, etc.), can be of advantage for improving ultrasound-based imaging, and or other imaging techniques.

### Example (e)

### Using the Ultrasound Pill to Improve siRNA Delivery

The ability to deliver siRNA to the oral cavity and/or to the digestive tract has been a major goal. However, the susceptibility of siRNA to degradation remains a major challenge. Using ultrasound to increase the permeability and/or to propel genes (such as siRNA) into the mucosal membrane can improve siRNA therapy.

### Example 2

### Experimental Validation—Enhancement of Permeability of GI tissue

The ability to enhance the permeability of the GI membrane by ultrasound was evaluated in the porcine small intestine. Tissue specimens from animals sacrificed within 24 hours of the experiment were mounted in Franz diffusion cells (PermeGear, Hellertown, Pa.), as described below.

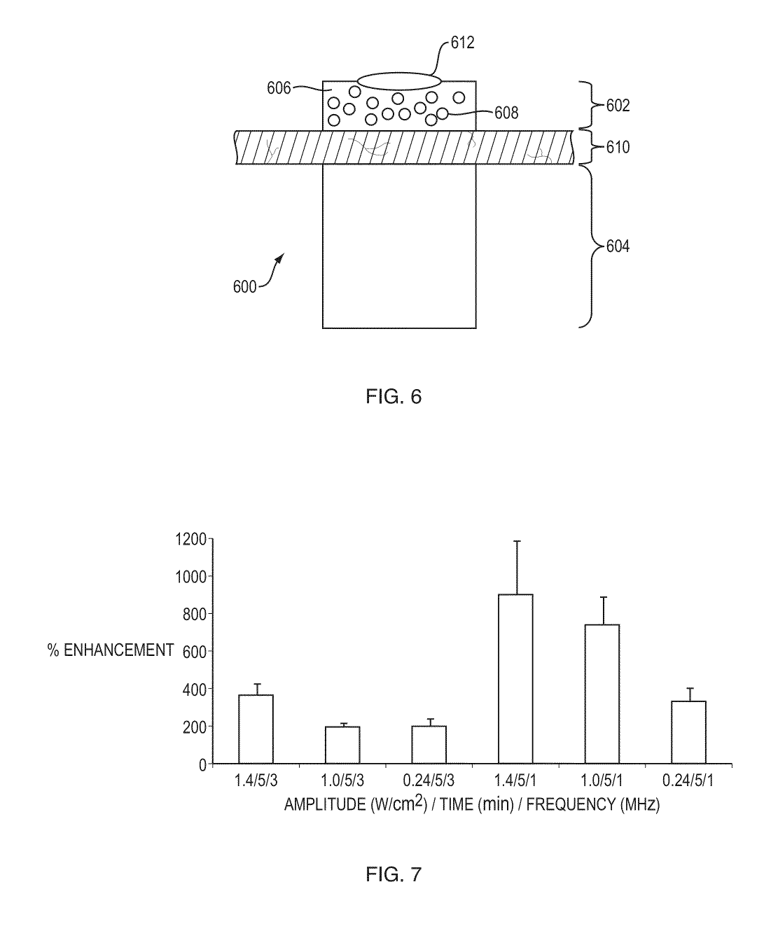

FIG. 6 is a schematic diagram illustrating the experimental system. Freshly harvested porcine small intestine tissue was placed in a Franz diffusion cell 600, which includes a donor cell or chamber 602 and an acceptor cell or chamber 604. The donor chamber 602 was loaded with phosphate buffered saline (PBS) 606 and a radio-labeled model compound 608, while the receiver chamber 604 contained PBS alone. Specimens of GI tissue 610 were positioned so that the luminal surface faced the ultrasound apparatus 612 at a distance of 1 mm (Enraf Nonius Sonopuls 490 equipped with a 15 mm diameter probe), simulating the in vivo scenario. The tissue was exposed to ultrasound, and the concentration of the delivered compound in the receiving cell and in the tissue was measured.

Through scintigraphy of the tissue and of the solution in the receiver chamber permeation enhancement was measured. For this, after each experiment, the tissue was removed from the diffusion cell 600 and washed thoroughly with PBS, to remove radio-labeled compounds that were not inside the tissue. Then, the tissue was dissolved (Souene 350, Perkin Elmer, USA), immersed in a scintillation fluid (HionicFluor, Perkin Elmer), and then measured for radiolabeled content with a scintillation counter (Tri Carb Scintillation Counter, Hewlett Packard). Similarly, the acceptor cell was measured for the radiolabeled content. All data are presented in comparison to sham samples—not treated by ultrasound.

### Effect of Ultrasonic Frequency and Amplitude

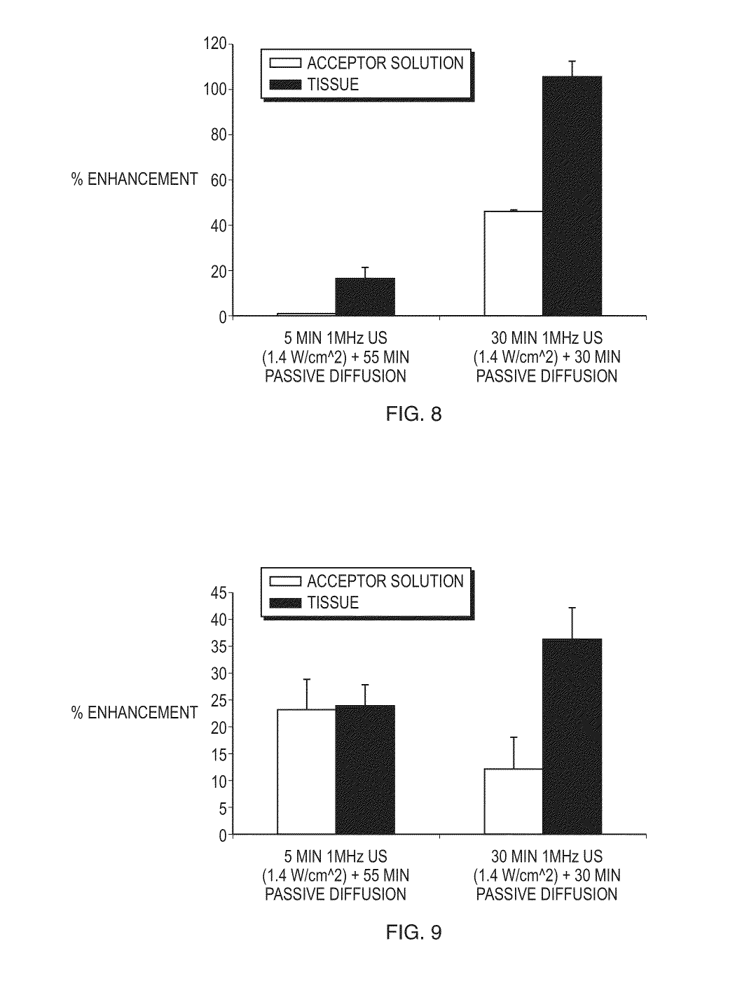

FIG. 7 is a graph illustrating enhancement of sucrose penetration into porcine small intestine due to exposure to 1-, or 3-MHz ultrasound at different amplitudes. Comparing the effect of 1- or 3-MHz ultrasound showed that the lower frequency was more effective in enhancing tissue permeability. For this, the donor cell was loaded with PBS enriched with ¹⁴C-labeled sucrose (Mw 342, American Radiolabeled Chemicals ARC, Saint Louis, Mo.)—a model compound simulating a small molecule drug. Ultrasound was applied for 5 minutes at the different frequencies (1- or 3-MHZ), and at different amplitudes (1.4, 1.0, or 0.24 W/cm²). Measuring the concentration of the radiolabeled molecule in the acceptor cell immediately after ultrasonic exposure showed that the lower frequency was more effective than the higher frequency in transporting the model compound across the tissue (FIG. 8, three right bars). Furthermore, these experiments showed that the higher the amplitude the more efficient the drug transport across the membrane (1.4>1.0>0.24).

Further penetration enhancement was evaluated using 1-MHz ultrasound across a wide range of compounds having Mw of 0.3-150 kDa. For all model compounds a significant penetration enhancement was measured.

### Protein-Scale Delivery

The donor cell was loaded with PBS enriched with ¹⁴C-labeled inulin (Mw 5000, ARC)—a model compound simulating a protein drug. Ultrasound was applied at two different irradiation profiles, which simulate the clearance kinetics of medium or high-Mw compounds in the GI tract:
1.) 5 minutes of ultrasonic irradiation at 1.4 W/cm², followed by 55 minutes of passive diffusion, or;
2.) 30 minutes of ultrasonic irradiation at 1.4 W/cm², followed by 30 minutes of passive diffusion.

FIG. 8 is a graph illustrating enhanced penetration of ¹⁴C-inulin into porcine small intestine measured after exposure to 1-MHz ultrasound (1.4 W/cm²) at different irradiation profiles. Black tiles represent the enhancement of inulin uptake in the tissue, while the white tiles represent the enhancement % of transport of inulin across the tissue. Thus, FIG. 8 presents the transport of the compound into the tissue itself or across the tissue and into the acceptor cell, at the two different irradiation profiles. As shown, the longer irradiation time was more effective in transporting inulin into or across the tissue.

### Macromolecule Delivery

The donor cell was loaded with PBS enriched with ¹⁴C-labeled dextrose (Mw 70,000, ARC)—a model compound simulating a macromolecule drug. Ultrasound was applied at two different irradiation profiles, which simulate the clearance kinetics of medium or high-Mw compounds in the GI tract:
1.) 5 minutes of ultrasonic irradiation at 1.4 W/cm², followed by 55 minutes of passive diffusion, or;
2.) 30 minutes of ultrasonic irradiation at 1.4 W/cm², followed by 30 minutes of passive diffusion.

FIG. 9 is a graph illustrating enhanced penetration of 70 kDa ¹⁴C-dextrose into porcine small intestine after exposure to 1-MHz ultrasound (1.4 W/cm²) at different irradiation profiles. Black tiles represent the enhancement of inulin uptake in the tissue, while the white tiles represent the enhancement % of transport of inulin across the tissue. Thus, FIG. 9 presents the transport of the compound into the tissue itself or across the tissue and into the acceptor cell, at the two different irradiation profiles. As shown, the longer irradiation time was more effective in transporting 70 kDa dextrose across the tissue.

To visualize the enhanced uptake of macromolecules to the small intestine by ultrasound, Alexa Fluor 700 goat anti rabbit IgG (Mw 150,000), a monoclonal antibody, was dissolved in the donor cell and the tissue was exposed to ultrasound (1 MHz, 1.4 W/cm²) for 30 min, followed by 30 min of passive diffusion.

### Example 3

### Tethered Ultrasound Device

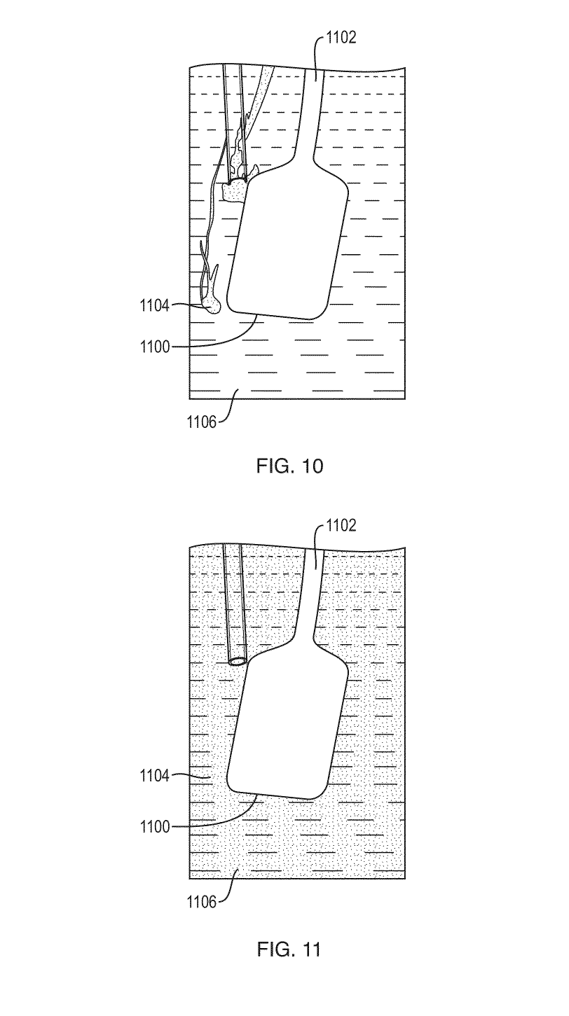

FIG. 10 is an image of an embodiment of an ultrasound device 1100 in a fluid. The device 1100 is tethered through a wire 1102 to a controller (not shown) to control operation of the device. FIG. 1 depicts the device in the “off” state, not emitting ultrasound. A model tracer (dye) 1104 has been injected into the fluid 1106. FIG. 11 is an image of the device 1100 showing the device in the “on” state, emitting ultrasound, illustrating mixing of the tracer and fluid. The device produces cavitation which propels the model tracer through the fluid.

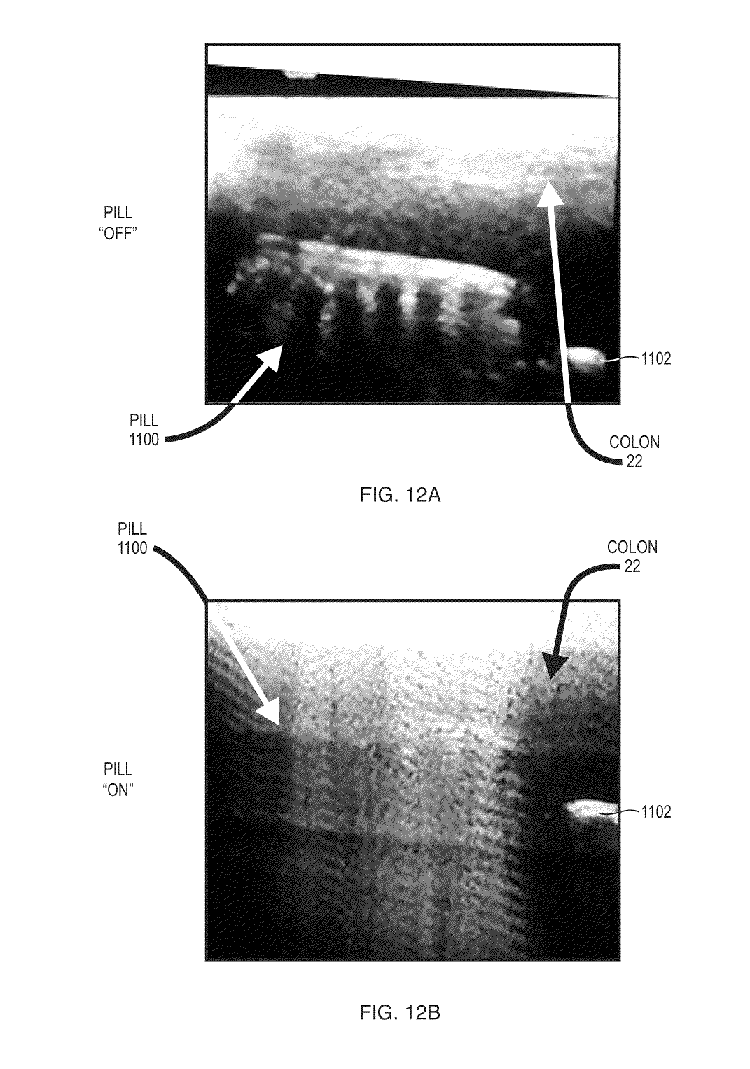

FIG. 12A is an image of the device 1100 of FIG. 10 in a colon recorded with an external ultrasound imaging apparatus. Only a portion of the colon 22 is visible, as is wire 1102. FIG. 12B is an image of the device of FIG. 10 illustrating the interference pattern induced by the ultrasound being emitted from the device 1100.

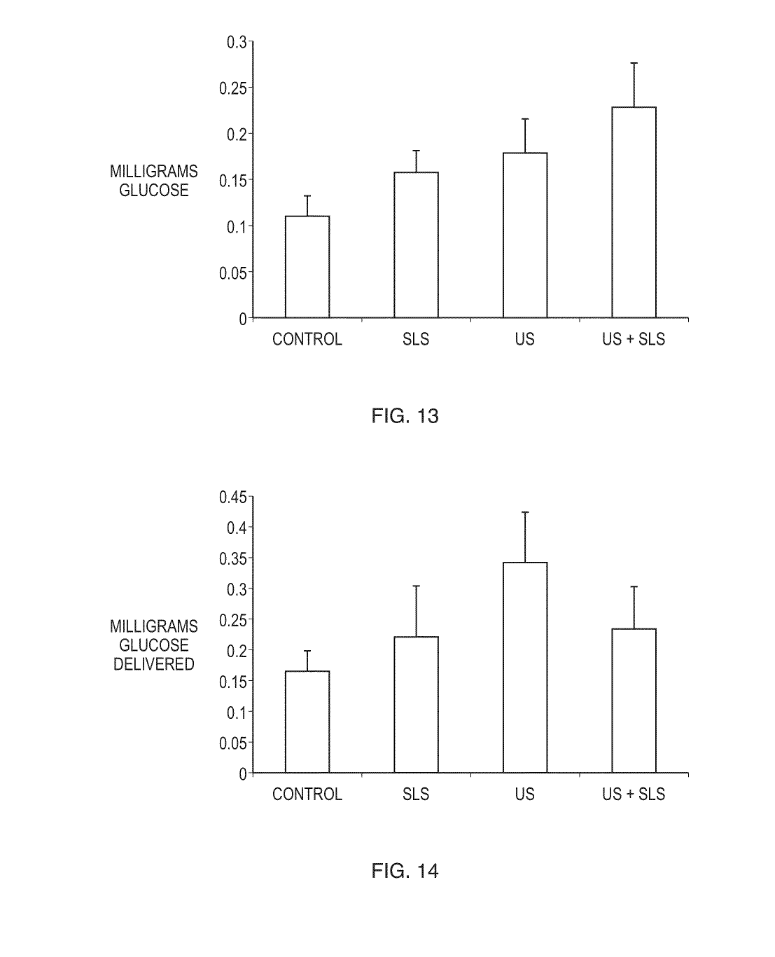

FIGS. 13 and 14 are a graph illustrating enhancement of delivery of a radioactive tracer (glucose) to tissue of the gastrointestinal tract using an ultrasound device, such as that shown in FIG. 12A. Ultrasonic waves at 600 kHz were applied for 3 minutes to gastrointestinal tissue in the presence (“SLS+US”) or absence (“US”) of the permeation enhancer sodium lauryl sulfate (SLS). The amount of glucose delivered without ultrasound and SLS (“Control”) and with SLS only (“SLS”) are also shown. FIG. 13 shows data obtained in the small intestine and FIG. 14 shows data obtained in the colon.

In the methods and devices described herein, the substance or compound to be delivered can be provided via one or more reservoirs. A reservoir can be a coating of a device, e.g., a coating applied to a housing of the device, or an internal reservoir or volume of the device. For example, the coating may be applied to the housing at a location where ultrasonic waves are emitted from the device. The reservoir can include polymeric material or material similar to the transdermal patches presently in use. The material can be sensitive to the ultrasound, as described in U.S. Appl. Ser. No. 06/936,000 entitled “Ultrasonically Modulated Polymeric Devices for Delivering Compositions” filed Nov. 28, 1986 by Joseph Kost and Robert S. Langer, now U.S. Pat. No. 4,779,806, or the material can release the substance to be delivered at a rate independent of the application of ultrasound. Many formulations are known to those skilled in the art which are safe for use internally and dissolve in the GI tract. Many biocompatible polymers can be used to form a polymeric matrix for the substance to be delivered, including both biodegradable and non-biodegradable polymers such as polyanhydrides, polylactic acid, polyglycolic acid, ethylene vinyl acetate copolymers, polypropylene, polyethylene. The release rate can also be manipulated by the form used to encapsulate the substance to be delivered. For example, the release rate from microcapsules is different from a slab containing the substance, even when made of the same material.

The methods and devices described herein can include small molecules. Examples of small molecules include organic compounds, organometallic compounds, inorganic compounds, and salts of organic, organometallic or inorganic compounds. Atoms in a small molecule are typically linked together via covalent and/or ionic bonds. The arrangement of atoms in a small organic molecule may represent a chain (e.g. a carbon-carbon chain), or may represent a ring containing carbon atoms, or, in some embodiments, a combination of carbon and heteroatoms. In some embodiments the small molecules are no more than about 5,000 daltons. For example, such small molecules can be no more than about 1000 daltons and, in some embodiments, are no more than about 750 daltons, for example, they can be less than about 500 daltons. Small molecules can be found in nature (e.g., identified, isolated, and/or purified) and/or produced synthetically (e.g., by organic synthesis and/or bio-mediated synthesis). See, e.g. Ganesan, Drug Discov. Today 7(1): 47-55 (January 2002); Lou, Drug Discov. Today, 6(24): 1288-1294 (December 2001), both of which are incorporated by reference in their entirety. Examples of naturally occurring small molecules include, but are not limited to, hormones, neurotransmitters, nucleotides, amino acids, sugars, lipids, and their derivatives.

In some embodiments, suitable dosages for small molecule substances that are administered to patients can be, for example from about 0.001 mg/kg to about 100 mg/kg, from about 0.01 mg/kg to about 100 mg/kg, from about 0.01 mg/kg to about 10 mg/kg, or from about 0.01 mg/kg to about 1 mg/kg body weight per treatment, e.g., per day.

Other substances that may be used with the devices described herein include polypeptides. Examples of polypeptides include any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a polypeptide can be free (e.g., unmodified) or protected with a suitable protecting group. Suitable protecting groups for amino and carboxyl groups, and methods for adding or removing protecting groups are known in the art. See, for example, Green and Wuts, “Protecting Groups in Organic Synthesis,” John Wiley and Sons, 1991. The functional groups of a polypeptide can also be derivatized (e.g., alkylated) using art-known methods.

An example polypeptide can include one or more modifications (e.g., amino acid linkers, acylation, acetylation, amidation, methylation, terminal modifiers (e.g., cyclizing modifications)), if desired. The polypeptide can also contain chemical modifications (e.g., N-methyl-α-amino group substitution). In addition, a polypeptide antagonist, which can be used with embodiments of the methods or devices disclosed herein, can be an analog of a known and/or naturally-occurring polypeptide, for example, a polypeptide analog having conservative amino acid residue substitution(s). These modifications can improve various properties of the polypeptide (e.g., solubility, binding), including its therapeutic efficacy.

It should be understood that the polypeptides can be linear, branched or cyclic, e.g., a peptide having a heteroatom ring structure that includes several amide bonds. In a particular embodiment, the peptide is a cyclic peptide. Such peptides can be produced by one of skill in the art using standard techniques.

The polypeptides can also encompass chimeric, or fusion, proteins that include all or a portion of a first protein operatively linked to all or a portion of a second, heterologous protein. “Operatively linked” indicates that the portions of the first protein and the heterologous protein are fused in-frame. The heterologous protein can be fused to the N-terminus or C-terminus of the first protein. For example, the fusion protein can be a GST-fusion protein in which the protein sequences are fused to the C-terminus of a GST sequence. Other types of fusion proteins include, but are not limited to, enzymatic fusion proteins, for example, β-galactosidase fusion proteins, yeast two-hybrid GAL fusion proteins, poly-His fusions, FLAG-tagged fusion proteins, GFP fusion proteins, and immunoglobulin (Ig) fusion proteins. Such fusion protein can facilitate purification (e.g., of a recombinant fusion protein). In certain host cells (e.g., mammalian host cells), expression and/or secretion of a protein can be increased by using a heterologous signal sequence. Therefore, in another embodiment, the fusion protein contains a heterologous signal sequence at its N-terminus.

It should be understood that fragments of proteins are intended to be compatible within the scope of embodiments of this invention.

In another embodiment, the device of the invention comprises peptidomimetic substances. For example, polysaccharides can be prepared that have the same functional groups as polypeptides. The binding moieties are the chemical atoms or groups which will react or form a complex (e.g., through hydrophobic or ionic interactions) with a target molecule, for example, a drug target. For example, the binding moieties in a peptidomimetic can be the same as those in a peptide or protein antagonist. The binding moieties can be an atom or chemical group which reacts with the receptor in the same or similar manner as the binding moiety in the peptide antagonist. Examples of binding moieties suitable for use in designing a peptidomimetic for a basic amino acid in a peptide include nitrogen containing groups, such as amines, ammoniums, guanidines and amides or phosphoniums Examples of binding moieties suitable for use in designing a peptidomimetic for an acidic amino acid include, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as, polyvinyl alcohol or polylactide).

Methods and devices according to embodiments of the present invention can also include substances that are nucleic acid molecules (e.g., oligonucleotides). Suitable nucleic acid molecules include aptamers, which are capable of binding to a particular molecule of interest (e.g., a drug target) with high affinity and specificity through interactions other than classic Watson-Crick base pairing (Tuerk and Gold, Science 249:505 (1990); Ellington and Szostak, Nature 346:818 (1990)).

Aptamers, like peptides generated by phage display or monoclonal antibodies (MAbs), are capable of specifically binding to selected targets and, through binding, block their targets' ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes.

The methods and devices described herein can be used to modulate or treat any disorder which can be treated locally or systemically.

In some embodiments, the methods and devices described herein can be used to treat any disorder which can be treated by a drug that can be administered through the GI tract. In some embodiments, the disorder is a disorder of the esophagus, including, but not limited to, esophagitis—(candidal), gastroesophageal reflux disease (gerd); laryngopharyngeal reflux (also known as extraesophageal reflux disease/eerd); rupture (Boerhaave syndrome, Mallory-Weiss syndrome); UES—(Zenker's diverticulum); LES—(Barrett's esophagus); esophageal motility disorder—(nutcracker esophagus, achalasia, diffuse esophageal spasm); esophageal stricture; and megaesophagus.

In some embodiments, the disorder is a disorder of the stomach, including but not limited to gastritis (e.g., atrophic, Menetrier's disease, gastroenteritis); peptic (i.e., gastric) ulcer (e.g., Cushing ulcer, Dieulafoy's lesion); dyspepsia; emesis; pyloric stenosis; achlorhydria; gastroparesis; gastroptosis; portal hypertensive gastropathy; gastric antral vascular ectasia; gastric dumping syndrome; HMFS (human mullular fibrilation syndrome).

In some embodiments, the disorder is a disorder of the small intestine, including but not limited to, enteritis (duodenitis, jejunitis, ileitis); peptic (duodenal) ulcer (curling's ulcer); malabsorption: celiac; tropical sprue; blind loop syndrome; Whipple's; short bowel syndrome; steatorrhea; milroy disease

In some embodiments, the disorder is a disorder of the small intestine, including but not limited to, both large intestine and small intestine enterocolitis (necrotizing); IBD (crohn's disease); vascular; abdominal angina; mesenteric ischemia; angiodysplasia; bowel obstruction: ileus; intussusception; volvulus; fecal impaction; constipation; and diarrhea (infectious).

In some embodiments, the disorder is a disorder of the small intestine, including but not limited to, accessory digestive glands disease; liverhepatitis (viral hepatitis, autoimmune hepatitis, alcoholic hepatitis); cirrhosis (PBC); fatty liver (Nash); vascular (hepatic veno-occlusive disease, portal hypertension, nutmeg liver); alcoholic liver disease; liver failure (hepatic encephalopathy, acute liver failure); liver abscess (pyogenic, amoebic); hepatorenal syndrome; peliosis hepatis; hemochromatosis; and Wilson's disease.

In some embodiments, the disorder is a disorder of the pancreas, including, but not limited to, pancreaspancreatitis (acute, chronic, hereditary); pancreatic pseudocyst; and exocrine pancreatic insufficiency.

In some embodiments, the disorder is a disorder of the large intestine, including but not limited to, appendicitis; colitis (pseudomembranous, ulcerative, ischemic, microscopic, collagenous, lymphocytic); functional colonic disease (IBS, intestinal pseudoobstruction/ogilvie syndrome); megacolon/toxic megacolon; diverticulitis; and diverticulosis.

In some embodiments, the disorder is a disorder of the large intestine, including but not limited to, gall bladder and bile ducts, cholecystitis; gallstones/cholecystolithiasis; cholesterolosis; Rokitansky-Aschoff sinuses; postcholecystectomy syndrome cholangitis (PSC, ascending); cholestasis/Mirizzi's syndrome; biliary fistula; haemobilia; and gallstones/cholelithiasis.

In some embodiments, the disorder is a disorder of the common bile duct (including choledocholithiasis, biliary dyskinesia).

Other disorders which can be treated with the methods and devices included herein include acute and chronic immune and autoimmune pathologies, such as systemic lupus erythematosus (SLE), rheumatoid arthritis, thyroidosis, graft versus host disease, scleroderma, diabetes mellitus, Graves' disease, Beschet's disease; inflammatory diseases, such as chronic inflammatory pathologies and vascular inflammatory pathologies, including chronic inflammatory pathologies such as sarcoidosis, chronic inflammatory bowel disease, ulcerative colitis, and Crohn's pathology and vascular inflammatory pathologies, such as, but not limited to, disseminated intravascular coagulation, atherosclerosis, giant cell arteritis and Kawasaki's pathology; malignant pathologies involving tumors or other malignancies, such as, but not limited to leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoides)); carcinomas (such as colon carcinoma) and metastases thereof; cancer-related angiogenesis; infantile haemangiomas; and infections, including, but not limited to, sepsis syndrome, cachexia, circulatory collapse and shock resulting from acute or chronic bacterial infection, acute and chronic parasitic and/or infectious diseases, bacterial, viral or fungal, such as a HIV, AIDS (including symptoms of cachexia, autoimmune disorders, AIDS dementia complex and infections).

Other disorders which can be treated with the methods and devices included herein include acute and chronic immune and autoimmune pathologies, inflammatory diseases, infections and malignant pathologies involving, e.g., tumors or other malignancies.

The dosage administered depends upon known factors such as the pharmacokinetic characteristics of the particular administered substance(s), the age, health, and weight of the patient; nature and the extent of the symptoms and the disorder, any other treatment, and frequency of treatment. In one embodiment, a dosage of the administered substance can be, for example about 0.01 to 100.0 milligrams per kilogram (mg/kg) of body weight, per day. In one embodiment, 1.0 to 5.0, e.g., 1 to 10 mg/kg per day can be given doses 1 to 5 times a day.

In some embodiments, treatment can be provided as a daily dosage of the administered substance of 0.1 to 100.0 mg/kg, such as 0.5, 1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or a combination thereof, for example, using doses of every 2, 4, 6, 8, 12 or 24 hours, or any combination thereof.

In some embodiments, polypeptides and proteins are administered in the devices and methods described herein. For example, in some embodiments, antibodies are used. Antibodies include polyclonal antibodies, monoclonal antibodies, human antibodies, humanized antibodies, engineered antibodies, and chimeric antibodies. Also included are fragments, regions or derivatives thereof. Antibodies can be of any immunoglobulin class, including IgG, IgM, IgE, IgA, GILD and any subclass thereof. Fragments can include, but are not limited to, F(ab′)2 Fragments, Fab′ Fragments, Fab Fragments, Fv Fragments, and Fc Fragments, as well as single chain fragments.

### REFERENCES

1. Kost, J., Mitragotri, S., Gabbay, R., Pishko, M & Langer, R. Transdermal monitoring of glucose and other analytes using ultrasound. Nat Medicine 6, 347-350 (2000).
2. Kinoshita, M, McDannold, N., Jolesz, F. A. & Hynynen, K Noninvasive localized delivery of Herceptin to the mouse brain by MRI-guided focused ultrasound-induced blood-brain barrier disruption. Proc Natl Acad Sci USA 103, 11719-23 (2006).
3. Mitragotri, S., Blankschtein, D. & Langer, R. Ultrasound-mediated transdermal protein delivery. Science 269, 850-3 (1995).
4. Tezel, A., Paliwal, S., Shen, Z. & Mitragotri, S. Low frequency ultrasound as a transcutaneous immunization adjuvant. Vaccine 23, 3800-7 (2005).
5. Henderson, P. W. et al. A portable high-intensity focused ultrasound device for noninvasive venous ablation. J Vasc Surg 51, 707-11 (2010).
6. Tang, H; Wang, C. C. l; Blankschtein, D.; Langer, R., An investigation of the role of cavitation in low frequency ultrasound-mediated transdermal drug transport. Pharm. Res. 2002, 19, (8), 1160-1169.
7. Rokhina, E. V.; Lens, P.; Virkutyte, l, Low-frequency ultrasound in biotechnology: state of the art. Trends in Biotechnology 2009, 27, (5), 298-306.
8. Mitragotri, Samir, Healing sound: the use of ultrasound in drug delivery and other therapeutic applications, Perspectives, 4, 255-260 (March 2005) www.nature.com/reviews/drugdisc
9. Polat, Baris E., Blankschtein, Daniel and Langer, Robert, Low-frequency sonophoresis: application to the transdermal delivery of macromolecules and hydrophilic drugs, Expert Opin. Drug Deliv. 7(12) 1415-1432 (2010).
10. U.S. Pat. No. 4,948,587, titled “Ultrasound Enhancement of Transbuccal Drug Delivery” by Joseph Kost, et al., issued: Aug. 14, 1990.
11. U.S. patent application Ser. No. 10/635,145, titled “Seryl transfer RNA synthetase polynucleotides and polypeptides and methods of use thereof” by Nancy H Hopkins, et al., filed Aug. 6, 2003, and published Jul. 22, 2004 as US 2004/0142440.
12. “Primary Care Medicine: Office Evaluation and Management of the Adult Patient,” Publisher: Lippincott Williams & Wilkins; 5 Har/Psc edition (Dec. 19, 2006).

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims. For example, an embodiment of an ultrasound device described herein could operate across a range of frequencies from about 20 kHz to about 50 MHz, or lower, or higher. Embodiments may also be used or modified to deliver a substance and/or apply ultrasound to a body lumen or cavity of the reproductive tract.

What is claimed is:

1. A method comprising:
providing a substance at a location in a gastrointestinal (GI) tract, excluding a buccal membrane, of a biological body; and
applying ultrasonic waves, having a frequency between about 20 kHz and about 10 MHz, at the location.

2.-10. (canceled)

11. The method according to claim 1, further comprising propelling the substance towards a surface of the GI tract with the ultrasonic waves.

12.-18. (canceled)

19. The method according to claim 1, further comprising tuning the ultrasonic waves to increase or decrease enzymatic activity in the GI tract while operating therein.

20.-28. (canceled)

29. The method according to claim 1, wherein applying the ultrasonic waves includes applying the ultrasonic waves at a frequency and intensity known to treat a medical condition through direct stimulus of the ultrasonic waves.

30. The method according to claim 29, wherein the ultrasonic waves applied include high intensity focused ultrasound (HIFU).

31. (canceled)

32. The method according to claim 1, further comprising:
delivering a device into the GI tract, the device comprising at least one ultrasound transducer and circuitry;
powering the at least one ultrasound transducer and circuitry from within the device; and
driving the at least one ultrasound transducer, using the circuitry, in a manner causing the at least one ultrasound transducer to emit ultrasonic waves to a medium within or of the GI tract.

33. The method according to claim 32, wherein the device includes a housing with holes defined therethrough and wherein providing the substance includes exposing the substance to a medium at the location via the holes.

34. The method according to claim 32, further comprising propelling the device within the GI tract with the ultrasonic waves emitted or other ultrasonic waves emitted by the device.

35. The method according to claim 32, wherein delivering the device includes rectally introducing the device into the GI tract.

36.-38. (canceled)

39. The method according to claim 32, further comprising positioning the device in the GI tract.

40. The method according to claim 39, wherein positioning the device includes employing a positioning mechanism to hold the device stationary.

41. (canceled)

42. The method according to claim 39, wherein positioning the device includes causing the device to adhere to tissue of the GI tract.

43.-44. (canceled)

45. A device comprising:
a housing configured to be delivered into a gastrointestinal (GI) tract of a biological body;
at least one ultrasound transducer, disposed within the housing, configured to emit ultrasonic waves, external from the housing, within a frequency range of from about 20 kHz to about 10 MHz to a medium within or of the GI tract;
circuitry, disposed within the housing, configured to drive the transducer; and
a power source, disposed within the housing, configured to power the transducer and the circuitry.

46.-59. (canceled)

60. The device according to claim 45, wherein the device is calibrated to emit ultrasonic waves to increase or decrease enzymatic activity in the body lumen.

61.-62. (canceled)

63. The device according to claim 45, wherein the device is configured to be positioned in the GI tract and held stationary using a positioning mechanism.

64. The device according to claim 63, wherein the positioning mechanism is a suture or an externally-placed magnet.

65. The device according to claim 45, wherein the device is configured to adhere to tissue of the GI tract.

66. The device according to claim 65, wherein the device includes a bioadhesive coating, and wherein the device is configured to adhere to the tissue of the GI tract through the use of the bioadhesive coating.

67. The device according to claim 65, wherein the device includes an adhesive releasable from a reservoir of the device.

68. (canceled)

69. The device according to claim 45, wherein the at least one ultrasound transducer is configured to emit the ultrasonic waves in the presence of a substance to be delivered at a location of the GI tract.

70. A method of treating inflammatory bowel disease, the method comprising:
providing a self-powered ultrasound device comprising at least one reservoir, each reservoir comprising a substance that includes a therapeutic molecule for treatment of inflammatory bowel disease;
releasing the substance from the at least one reservoir into the gastrointestinal tract in the vicinity of target tissue for localized delivery of the substance; and
emitting ultrasonic waves in a frequency range of from about 20 kHz to about 10 MHz with the ultrasound device to increase passage of the substance through a surface of the target tissue.

71.-74. (canceled)
